(19) United States
(12) Patent Application Publication
(10) Pub. No.: US 2026/0191792 A1
(43) Pub. Date: Jul. 9, 2026

(54) CYCLODEXTRIN ENCAPSULATED PROBIOTIC STRAINS

(71) Applicant: Zognition, LLC, Highland, MI (US)

(72) Inventor: Peter Angia Pham, Spring, TX (US)

(73) Assignee: Zognition, LLC, Highland, MI (US)

(21) Appl. No.: 19/011,260

(22) Filed: Jan. 6, 2025

(51) Int. Cl. A61K 9/50; A61K 35/00; A61K 35/741; A61K 35/742; A61K 35/744; A61K 35/745; A61K 35/747

(52) U.S. Cl. CPC A61K 9/5036; A61K 35/741; A61K 35/742; A61K 35/744; A61K 35/745; A61K 35/747; A61K 2035/115

(57) ABSTRACT

The present disclosure teaches methods, materials, and structures for encapsulating probiotic microorganisms in a biocompatible encapsulation compound. The encapsulation compound may be a cyclodextrin macrocyclic molecule that has a hollow, cone-shaped, central cavity. Probiotic microorganisms are then encapsulated partially, or completely, inside of the hollow, central cavity of the cyclodextrin molecule to make a cyclodextrin inclusion complex. The resulting encapsulated probiotic assembly provides enhanced stability and bioavailability of the probiotic microorganisms during storage and after ingestion, as well as offering protection against environmental stressors, such as heat, moisture, and oxygen. The sugar-like cyclodextrin encapsulant material may also serve a prebiotic function of supporting beneficial gut flora proliferation and improved probiotic viability in the gastrointestinal tract. The cyclodextrin macrocyclic molecule may be an α-cyclodextrin, β-cyclodextrin, and/or γ-cyclodextrin molecule, and/or combinations thereof. Other biocompatible encapsulation compounds may be used, including polysaccharides, liposomes, alginates, chitosans, gelatins, and polyvinyl alcohols.

### INTRODUCTION

This disclosure relates to methods, materials, and structures for (1) encapsulating one or more probiotic strains in a hollow, cyclodextrin-based structure, and (2) for administering these encapsulated probiotic strains to a human or animal host. Cyclodextrins (CDs), as natural oligosaccharides, are used for their biodegradability, biocompatibility, non-toxicity, and internal hydrophobic and external hydrophilic structural features.

Cyclodextrins are cyclic oligosaccharides with hydroxyl groups on the outer surface and a hollow cavity in the center. Their outer surface is hydrophilic, and, hence, cyclodextrins are usually soluble in water. However, the cavity has a hydrophobic and lipophilic character. The most common cyclodextrins are α-cyclodextrin, β-cyclodextrin and γ-cyclodextrin, consisting of 6, 7 and 8 α-1,4-linked glucopyranose linked sub-units, respectively. The number of these linked sub-units determines the size of the cavity. Cyclodextrins are capable of forming inclusion complexes with a wide variety of hydrophobic molecules by taking up (i.e., encapsulating) an entire guest molecule, or some portion of it, into the cavity. The stability of the inclusion complex thus formed depends on how well the guest molecule fits into the cyclodextrin cavity.

Cyclodextrins comprise a family of cyclic oligosaccharides, consisting of a macrocyclic ring of glucose sub-units joined by α-1,4 glycosidic bonds. Cyclodextrins are produced from starch by enzymatic conversion. Cyclodextrins are linked in a macrocyclic way that form a cone-shaped, hollow structure having a hydrophilic outer surface and an inner hydrophobic (and lipophilic) central cavity. Inclusion complexes are formed when compounds of interest (i.e., “guest molecules”) enter the hydrophobic cavity in an energetically favorable reaction. This central cavity gives cyclodextrins the ability to serve as microencapsulation substrates for a variety of compounds, including pharmaceutical, nutraceutical, cosmetic, and food ingredients.

Cyclodextrins are composed of five or more α-D-glucopyranoside units linked 1→4, as in amylose (a fragment of starch). Typical cyclodextrins contain a number of glucose monomers ranging from six to eight units in a ring, creating a hollow, cone-shaped structure, comprising: six glucose sub-units in α-cyclodextrin; seven glucose sub-units in β-cyclodextrin; and eight glucose sub-units in γ-cyclodextrin. The largest, well-characterized cyclodextrin contains thirty-two 1,4-anhydroglucopyranoside units.

As used herein, and in the appended claims, the term “cyclodextrin” refers to a macrocyclic dextrin molecule that is formed by enzyme conversion of starch. Specific enzymes, e.g., various forms of cyclo-glycosyl transferase (CGTase), may be used to break down helical structures that occur in starch to form specific cyclodextrin molecules having three-dimensional, polyglucose rings with, e.g., 6, 7, or 8 glucose sub-units. For example, α-CGTase can convert starch to α-cyclodextrin having 6 glucose sub-units, β-CGTase can convert starch to β-cyclodextrin having 7 glucose sub-units, and γ-CGTase can convert starch to γ-cyclodextrin having 8 glucose sub-units. Cyclodextrins include, but are not limited to, at least one of α-cyclodextrin, β-cyclodextrin, and γ-cyclodextrin, and/or combinations thereof.

Cyclodextrin-based molecules are not known to have toxic effects. The safety of cyclodextrin is supported by extensive scientific data and regulatory approvals worldwide. The FDA classifies cyclodextrins as one of the GRAS (Generally Recognized as Safe) products based on the research and historical data.

The three-dimensional, cyclic structure (i.e., macrocyclic structure) of a cyclodextrin molecule includes an external portion, which includes both primary and secondary hydroxyl groups, and which are hydrophilic. The cyclodextrin molecule also includes a three-dimensional, generally “cone-shaped” cavity that comprises carbon atoms, hydrogen atoms, and ether linkages, which are hydrophobic. The hydrophobic cavity (i.e. “cage”) of the cyclodextrin molecule may act as a host and hold a variety of guest molecules to form a cyclodextrin inclusion complex.

Cyclodextrin's structure, with a hydrophobic cone-shaped cavity and hydrophilic exterior, enables it to encapsulate a wide variety of compounds based on their partition coefficient (P), which characterizes their hydrophilic or hydrophobic nature. Compounds with positive log(P) values (indicating hydrophobicity) will preferentially bind within cyclodextrin's cavity, forming stable inclusion complexes in an aqueous environment. This binding occurs because the hydrophobic guest molecule is thermodynamically driven to avoid water by associating with the similarly hydrophobic interior of cyclodextrin.

Moreover, cyclodextrin can also encapsulate hydrophilic and amphipathic compounds due to its adaptable binding environment. Amphipathic molecules, which have both hydrophobic and hydrophilic regions, can position their hydrophobic parts within the cyclodextrin cavity while their hydrophilic parts interact with the aqueous surroundings. In some embodiments, when it is beneficial to complex multiple guest molecules, each guest can be encapsulated separately to maximize encapsulation efficiency and target a broad range of compounds effectively.

Because the cone-shaped cavity of cyclodextrin is hydrophobic relative to its exterior surface, guest molecules having positive log(P) values (particularly, relatively large positive log(P) values) will be readily encapsulated in cyclodextrin and form stable cyclodextrin inclusion complexes in an aqueous environment. This happens because the guest molecule will thermodynamically and prefer to energetically bind inside (completely or partially inside) to the cyclodextrin cavity in an aqueous environment. In some embodiments, when it is desired to complex more than one guest molecule, each guest molecule can be encapsulated separately to maximize the efficiency of encapsulating the guest of interest.

### SUMMARY

The present disclosure teaches methods, materials, and structures for encapsulating probiotic microorganisms in a biocompatible encapsulation compound. The encapsulation compound may be a cyclodextrin macrocyclic molecule that has a hollow, cone-shaped, central cavity. Probiotic microorganisms are then encapsulated partially, or completely, inside of the hollow, central cavity of the cyclodextrin molecule to make a cyclodextrin inclusion complex. The resulting encapsulated probiotic assembly provides enhanced stability and bioavailability of the probiotic microorganisms during storage and after ingestion, as well as offering protection against environmental stressors, such as heat, moisture, and oxygen. The sugar-like cyclodextrin encapsulant material may also serve a prebiotic function of supporting beneficial gut flora proliferation and improved probiotic viability in the gastrointestinal tract. The cyclodextrin macrocyclic molecule may be an α-cyclodextrin, β-cyclodextrin, and/or γ-cyclodextrin molecule, and/or combinations thereof. Other biocompatible encapsulation compounds may be used, including polysaccharides, liposomes, alginates, chitosans, gelatins, and polyvinyl alcohols.

In an embodiment, a method of encapsulating one or more probiotic microorganisms includes: combining one or more probiotic microorganisms with a solution, a suspension, or a medium comprising a biocompatible encapsulation compound, and encapsulating the probiotic microorganisms at least partially within the biocompatible encapsulation compound to form an encapsulated probiotic assembly.

The encapsulated probiotic assembly may be processed into a stable form suitable for oral administration to a human or animal host. The method may also include process steps for enhancing the stability, bioavailability, or targeted delivery of the encapsulated probiotics, such as cross-linking, freeze-drying, or coating with a protective polymer. The biocompatible encapsulation compound may be selected from, but is not limited to, a polysaccharide, liposome, alginate, chitosan, gelatin, or polyvinyl alcohol, and/or combinations thereof.

In another embodiment, the biocompatible encapsulation compound includes a cyclodextrin molecule, which when combined with a probiotic microorganism forms a cyclodextrin inclusion complex. The cyclodextrin molecule may be an α-cyclodextrin, β-cyclodextrin, or γ-cyclodextrin molecule, and may be derivatives of cyclodextrin. The probiotic microorganisms may include, but is not limited to: Lactobacillus spp., Bifidobacterium spp., Saccharomyces spp., Escherichia spp., Streptococcus spp., Bacillus spp., Enterococcus spp., Propionibacterium spp., Clostridium spp., Akkermansia spp., Faecalibacterium spp., Roseburia spp., Veillonella spp., Pediococcus spp., Leuconostoc spp., Lactococcus spp., Bacteroides spp., Eubacterium spp., Ruminococcus spp., Blautia spp., Parabacteroides spp., Desulfovibrio spp., Allobaculum spp., Megasphaera spp., Prevotella spp., Oscillibacter spp., Phascolarctobacterium spp., Coprococcus spp., Anaerostipes spp., Butyrivibrio spp., Collinsella spp., Dorea spp., Alistipes spp., Lactonifactor spp., Methanobrevibacter spp., Oscillospira spp., Sutterella spp., Eggerthella spp., Clostridioides spp., Turicibacter spp., Flavonifractor spp., Dialister spp., Bilophila spp., Bulleidia spp., lactic acid bacteria, spore-forming bacteria, yeast-based probiotics, genetically engineered probiotic strains, methanogens, commensal gut bacteria, and combinations thereof. The encapsulated probiotic microorganisms may be dried and stabilized by using freeze-drying, spray-drying, vacuum-drying, cross-linking, or coating with a protective polymer to make a powder form.

In some embodiments, the encapsulated probiotic assembly is formed into one or more delivery forms, including, but not limited to: capsules, powders, granules, suspensions, tablets, gels, sprays, gummies, or liquid delivery forms. Furthermore, an effective amount of the selected delivery form(s) of the encapsulated probiotic assembly may be administered to a human or animal host via, for example, oral, nasal, rectal, or other suitable routes of administration. The administered encapsulated probiotic assembly survives passage through the upper GI tract, including but not limited to: the esophagus, stomach, and duodenum of a human or animal host without prematurely releasing the probiotic microorganisms before the encapsulated probiotic assembly enters the lower GI tract such as the jejunum, ileum, colon or cecum and large intestine of a human or animal host to ensure optimal delivery and functionality within the host.

In an embodiment,, the encapsulated probiotic assembly provides enhanced stability and bioavailability of the probiotic microorganisms during storage and after ingestion; as well as offering protection against environmental stressors including but not limited to heat, moisture, pH variability, mechanical stress, and/or oxidative conditions. The encapsulation may include additional stabilizers, coatings, or other agents to increase resistance to environmental challenges and extend the shelf life of the product.

In another embodiment, a method of encapsulating probiotic microorganisms includes combining the probiotic microorganisms with one or more cyclodextrin molecules through one or more encapsulation techniques including, but not limited to co-precipitation, lyophilization, freeze-drying, spray-drying, vacuum-drying, kneading, solvent evaporating, nanoparticle forming, and/or combination thereof, which then forms a cyclodextrin inclusion complex that at least partially, or completely, encapsulates the probiotic microorganisms.

In another embodiment, the cyclodextrin molecule may serve one or more multiple functions including, but not limited to, acting as a prebiotic agent that promotes growth and metabolic activity of the one or more probiotic microorganisms when released from the cyclodextrin inclusion complex inside of a human or animal host. This prebiotic activity may enhance clinical efficacy of the probiotics for a wide range of applications including, but not limited to treating or managing dry eye disease, cardiovascular health, diabetes, dementia, obesity, skin health, oral health, wound care, and/or combinations thereof. Additionally, stabilizers, excipients, or active ingredients may be incorporated during encapsulation to further enhance the functionality or therapeutic potential of the inclusion complex.

In still another embodiment, a composition includes a pharmaceutically acceptable carrier and an encapsulated probiotic assembly combined with the pharmaceutically acceptable carrier, where the biocompatible encapsulation compound is selected from the group consisting of cyclodextrins, derivatized cyclodextrins, liposomes, polysaccharides, alginate, chitosan, gelatin, polyvinyl alcohol, and/or combinations thereof; and the composition includes one or more probiotic microorganisms encapsulated at least partially within the biocompatible encapsulation compound. The encapsulated probiotic assembly provides at least one of controlled release, enhanced stability, and targeted delivery of the probiotic microorganisms.

In an embodiment, the composition includes a cyclodextrin molecule and at least one probiotic microorganism disposed at least partially, or completely, inside of the cyclodextrin molecule. The encapsulated probiotic assembly may provide protection from environmental stressors and ensures targeted release in a lower gastrointestinal tract.

The composition may further include a pharmaceutically acceptable carrier or excipient. In some embodiments, the composition is suitable for oral administration or other delivery routes to a human or animal host. The administered encapsulated probiotic assembly is designed to survive passage through the upper Gastrointestinal (GI) tract (e.g., esophagus, stomach, and duodenum) of a human or animal host without prematurely releasing the probiotic microorganisms, before the encapsulated probiotic assembly enters the lower GI tract (e.g., jejunum, ileum, and large intestine) of a human or animal host. This targeted delivery promotes the functional benefits of the probiotics and enhances their therapeutic efficacy for various applications

In an embodiment, an encapsulated probiotic assembly, such as a cyclodextrin inclusion complex, may have a probiotic microorganism-to-cyclodextrin ratio, R, that ranges from about 0.1:1 to about 5:1. In other embodiments, the ratio, R, may range from about 0.2:1 to about 2:1. In other embodiments, the ratio, R, may range from about 0.5:1 to 1:1. The cyclodextrin inclusion complex may be in the form of particles or assemblies, including but not limited to: nanoparticles having a dimension ranging from about 1 nm to about 100 nm. Alternatively, the cyclodextrin inclusion complex may be in the form of particles or assemblies, including, but not limited to, nanoparticles having a dimension ranging from about 1 nm to about 10 nm.

In an embodiment, the cyclodextrin molecule may be derivatized or modified with one or more functional groups including, but not limited to: methyl, ethyl, acetyl, hydroxypropyl, sulfonate, or carboxymethyl groups to enhance one of more of solubility, stability, bioavailability, or targeted delivery properties. In some embodiments, the cyclodextrin inclusion complex may incorporate additional agents, such as stabilizers, active ingredients, or excipients, to further enhance functionality for specific therapeutic applications.

In an embodiment, the encapsulated probiotic assembly includes a porous, interconnected, cyclodextrin cage comprising a plurality of interstitial spaces filled with one or more cyclodextrin inclusion complexes including one or more probiotic microorganisms. The porous, interconnected, cyclodextrin cage may have a honeycomb buckyball shape. The cage may be made of one or more polymerized cyclodextrin molecules.

### BRIEF DESCRIPTION OF THE DRAWINGS

FIG. 1 shows a schematic chemical structure of an α-cyclodextrin macrocyclic molecule comprising six glucose sub-units.

FIG. 2 shows a schematic chemical structure of a β-cyclodextrin macrocyclic molecule comprising seven glucose sub-units.

FIG. 3 shows a schematic chemical structure of a γ-cyclodextrin macrocyclic molecule comprising eight glucose sub-units.

FIG. 4A shows measured dimensions of a three-dimensional, hollow cone-shaped structure of the α-cyclodextrin molecule.

FIG. 4B shows measured dimensions of a three-dimensional, hollow cone-shaped structure of the β-cyclodextrin molecule.

FIG. 4C shows measured dimensions of a three-dimensional, hollow cone-shaped structure of the γ-cyclodextrin molecule.

FIG. 5 shows a three-dimensional, solid-shaded volumetric representation of an example of a hollow, macrocyclic β-cyclodextrin molecule.

FIG. 6A shows a schematic representation of an example of a three-dimensional, hollow, cone-shaped structure of a β-cyclodextrin molecule.

FIG. 6B shows a chemical structure of an example of the repeating glucose sub-unit of a β-cyclodextrin molecule shown in FIG. 6A.

FIG. 7 shows a three-dimensional, schematic chemical representation of an example of a hollow, cone-shaped macrocyclic γ-cyclodextrin molecule with a sidewall and multiple protruding OH hydroxyl moieties located on primary and secondary ends of the γ-cyclodextrin molecule.

FIG. 8 shows a three-dimensional structural representation of an example of a hollow, cone-shaped, cyclodextrin molecule with a hydrophilic outer surface and a hydrophobic inner cavity.

FIG. 9 shows a three-dimensional schematic representation of an example of a method of encapsulating a guest molecule in a generic, hollow, cone-shaped, cyclodextrin molecule to form an encapsulated probiotic assembly.

FIG. 10A shows a three-dimensional schematic representation of an example of a 1:1 configuration for encapsulating (either entirely or partially) a single guest molecule in a single, generic, hollow, cone-shaped, cyclodextrin molecule.

FIG. 10B shows a three-dimensional schematic representation of an example of a 1:2 configuration for encapsulating (either entirely or partially) a pair of guest molecules in a single, generic, hollow, cone-shaped, cyclodextrin molecule.

FIG. 10C shows a three-dimensional schematic representation of an example of a 2:1 configuration for encapsulating (either entirely or partially) a single guest molecule in an opposing pair of generic, hollow, cone-shaped, cyclodextrin molecules.

FIG. 10D shows a three-dimensional schematic representation of an example of a 2:2 configuration for encapsulating (either entirely or partially) a pair of guest molecules in an opposing pair of generic, hollow, cone-shaped, cyclodextrin molecules.

FIG. 11 shows a three-dimensional schematic representation of an example of an encapsulated probiotic assembly comprising multiple guest molecules encapsulated in a spherical, biocompatible structural cage.

### DETAILED DESCRIPTION OF THE DISCLOSURE

As used herein, and in the appended claims, the term “guest” and/or “guest molecule” refers to any molecule or microorganism in which at least a portion thereof is held or captured within the three-dimensional cavity present inside of a cyclodextrin molecule. The terms “cyclodextrin” and “generic cyclodextrin” include: α-cyclodextrin, β-cyclodextrin, γ-cyclodextrin, and combinations thereof.

As used herein, the term “cyclodextrin inclusion complex” refers to a chemical complex that is formed by encapsulating at least a portion of one or more guest molecules with one or more cyclodextrin molecules (i.e., encapsulation on a molecular level) by capturing and holding a guest molecule partially or completely within the three-dimensional cyclodextrin cavity. The guest molecule may be held in position by Van der Waal forces within the cavity by hydrogen bonding and/or hydrophilic-hydrophobic interactions. The guest molecule may be released from the cavity when the cyclodextrin inclusion complex is dissolved by a solvent (e.g., water). Cyclodextrin inclusion complexes are also referred to herein as “guest-cyclodextrin complexes.” The terms “probiotic microorganisms” and “probiotic strains” are used interchangeably herein. The term “encapsulated probiotic assembly” broadly includes a “cyclodextrin inclusion complex”. A “guest molecule” may include one or more probiotic microorganisms. The term “probiotic bacteria microorganisms” and “probiotic strains” are used interchangeably. The term “upper gastrointestinal (GI) tract” includes at least one of an esophagus, stomach, and duodenum. The term “lower gastrointestinal (GI) tract” includes the at least one of a jejunum, ileum, cecum, colon, and large intestine of a human or animal host.

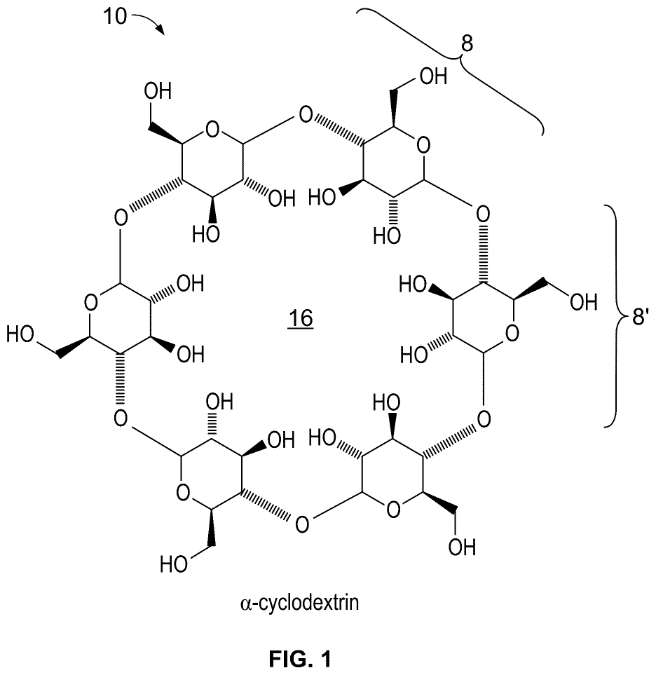

FIG. 1 shows a schematic chemical structure of an α-cyclodextrin macrocyclic molecule 10 comprising six glucose sub-units 8, 8′, etc. Hollow cavity 16 is located inside of α-cyclodextrin molecule 10.

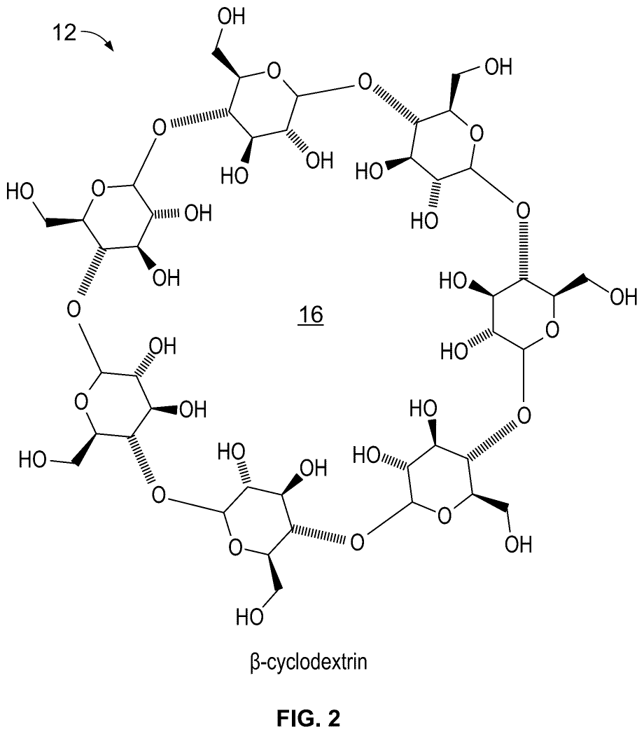

FIG. 2 shows a schematic chemical structure of a β-cyclodextrin macrocyclic molecule 12 comprising seven glucose sub-units 8, 8′, etc. Hollow cavity 16 is located inside of β-cyclodextrin molecule 12.

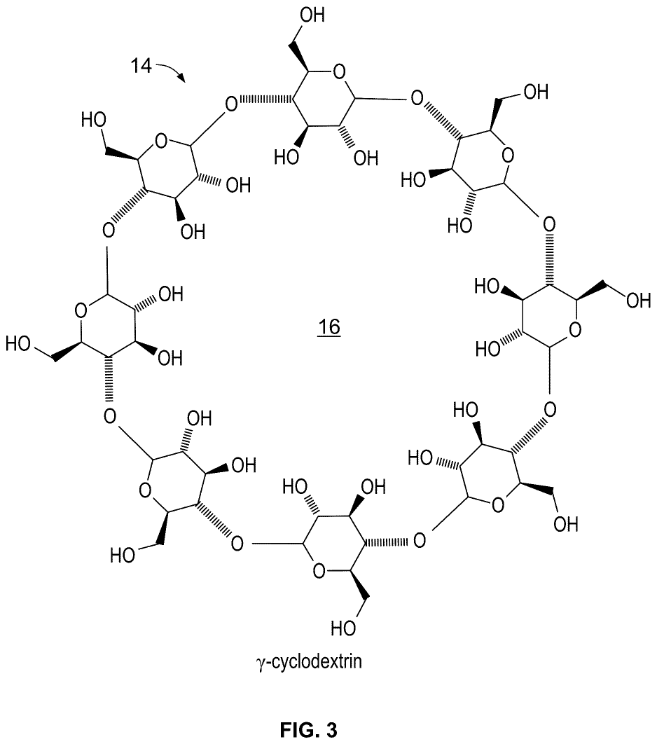

FIG. 3 shows a schematic chemical structure of a γ-cyclodextrin macrocyclic molecule 14 comprising eight glucose sub-units. Hollow cavity 16 is located inside of γ-cyclodextrin molecule 14.

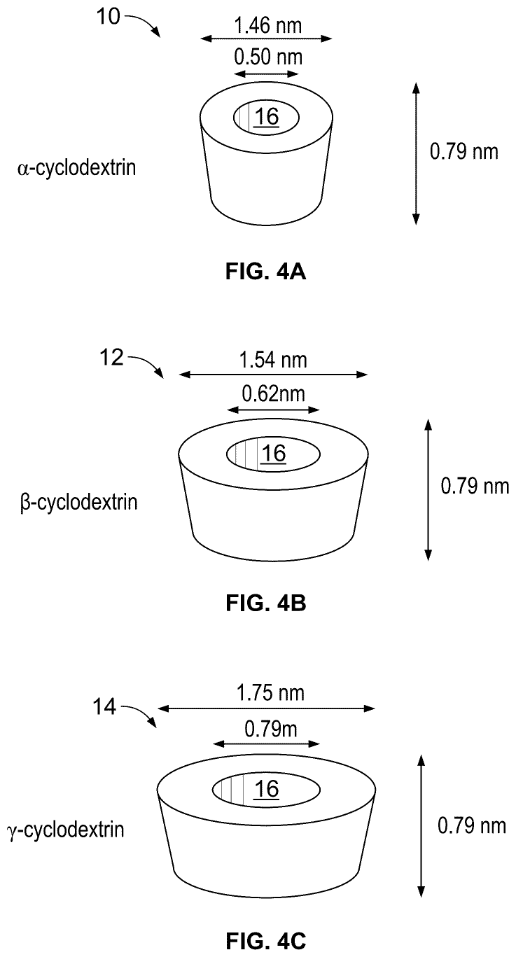

FIG. 4A shows measured dimensions (in nm) of the three-dimensional, hollow cone-shaped structure of an α-cyclodextrin molecule 10. The outer diameter of molecule 10 is about 1.46 nm and the inner diameter is about 0.50 nm.

FIG. 4B shows measured dimensions (in nm) of the three-dimensional, hollow cone-shaped structure of a β-cyclodextrin molecule 12. The outer diameter of molecule 12 is about 1.54 nm and the inner diameter is about 0.62 nm.

FIG. 4C shows measured dimensions (in nm) of the three-dimensional, hollow cone-shaped structure of a γ-cyclodextrin molecule 14. The outer diameter of molecule 14 is about 1.75 nm and the inner diameter is about 0.79 nm.

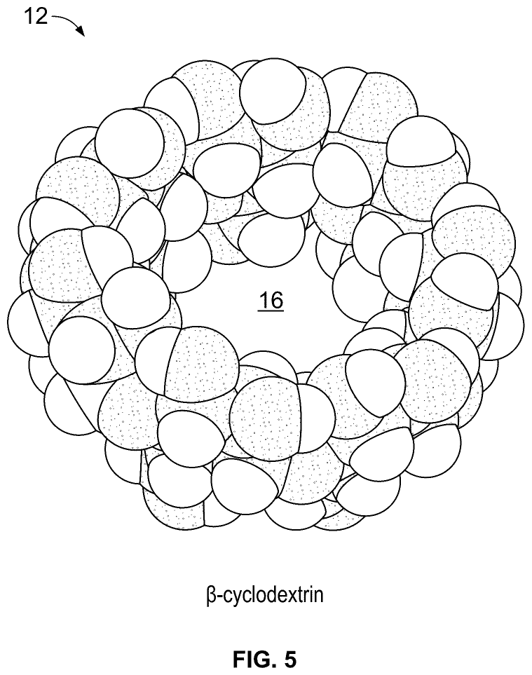

FIG. 5 shows a three-dimensional, solid-shaded volumetric representation of an example of a hollow, macrocyclic β-cyclodextrin molecule 12. Hollow cavity 16 is located inside of β-cyclodextrin molecule 12.

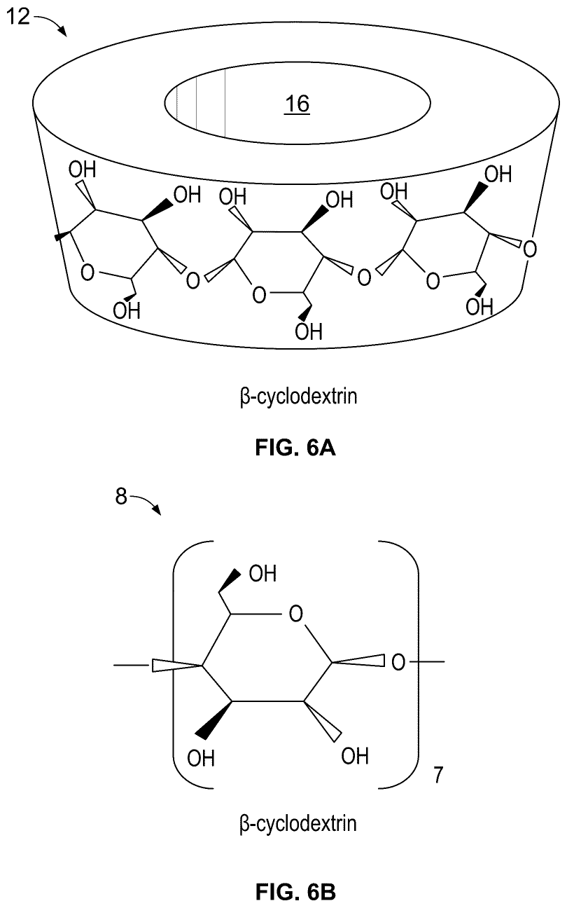

FIG. 6A shows a schematic representation of the three-dimensional, hollow cone-shaped structure of an example of a β-cyclodextrin molecule 12. Hollow cavity 16 is located inside of β-cyclodextrin molecule 12.

FIG. 6B shows a chemical structure of an example of the repeating glucose sub-unit 8 of the β-cyclodextrin molecule 12 shown in FIG. 6A.

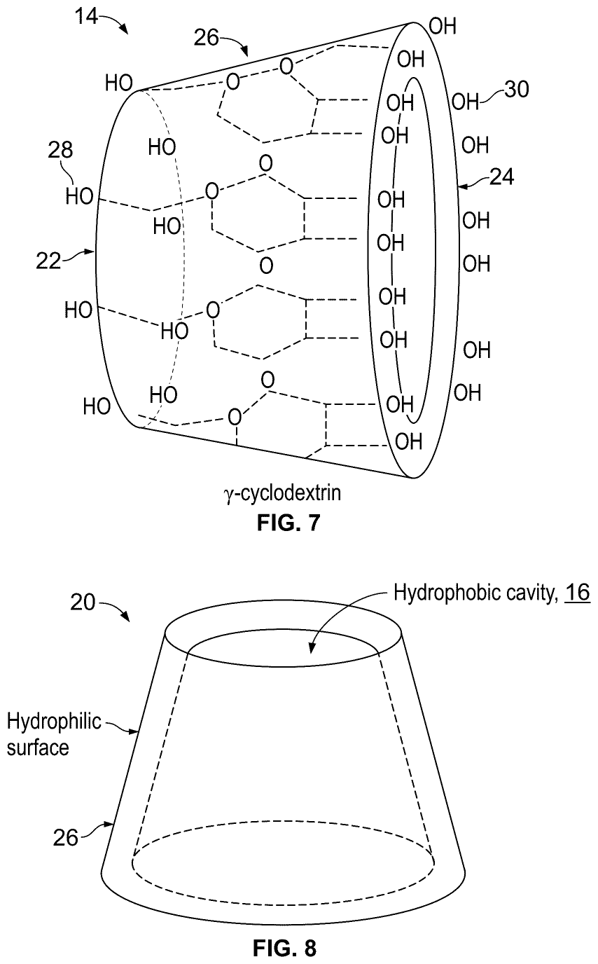

FIG. 7 shows a three-dimensional, schematic chemical representation of a hollow, cone-shaped macrocyclic γ-cyclodextrin molecule 14 with sidewall 26 and protruding OH hydroxyl moieties 28 and 30 located on primary and secondary ends 22 and 24, respectively of γ-cyclodextrin molecule 14.

FIG. 8 shows a three-dimensional structural representation of an example of a generic, hollow, cone-shaped, cyclodextrin molecule 20 with a hydrophilic outer surface 26 and a hydrophobic inner cavity 16. Inner cavity 16 is also lipophilic.

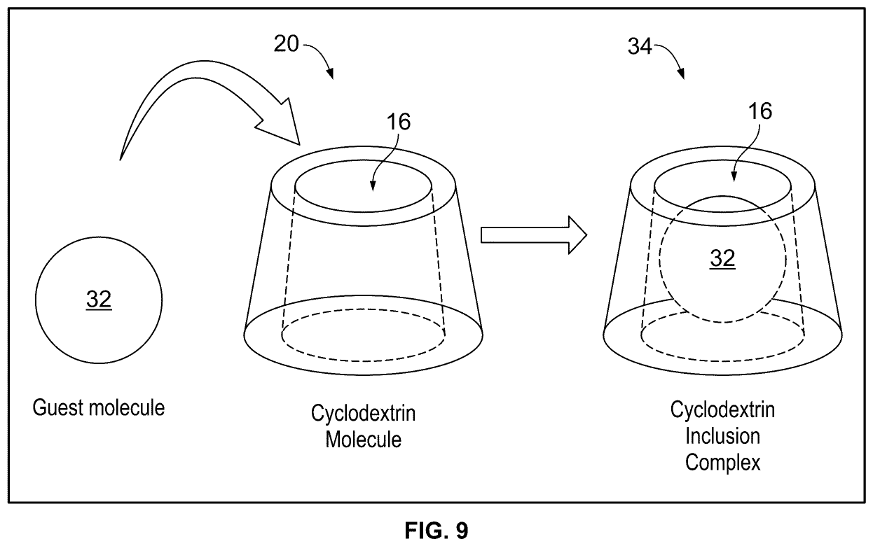

FIG. 9 shows a three-dimensional schematic representation of an example of a method of encapsulating a guest molecule 32 in a generic, hollow, cone-shaped, cyclodextrin molecule 20 to form an encapsulated probiotic assembly 34 In this example, guest molecule 32 is illustrated as being completely encapsulated inside of cavity 16 in cyclodextrin molecule 20. Alternatively, guest molecule 32 may be partially disposed inside of cavity 16. Guest molecule 32 may comprise one or more strains of probiotic microorganisms (not illustrated).

The probiotic microorganisms may be selected from a list comprising: lactic acid bacteria, spore-forming bacteria, yeast-based probiotics, genetically engineered probiotic strains, methanogens, commensal gut bacteria, and/or combinations thereof, including, but not limited to: Lactobacillus spp., Bifidobacterium spp., Saccharomyces spp., Escherichia spp., Streptococcus spp., Bacillus spp., Enterococcus spp., Propionibacterium spp., Clostridium spp., Akkermansia spp., Faecalibacterium spp., Roseburia spp., Veillonella spp., Pediococcus spp., Leuconostoc spp., Lactococcus spp., Bacteroides spp., Eubacterium spp., Ruminococcus spp., Blautia spp., Parabacteroides spp., Desulfovibrio spp., Allobaculum spp., Megasphaera spp., Prevotella spp., Oscillibacter spp., Phascolarctobacterium spp., Coprococcus spp., Anaerostipes spp., Butyrivibrio spp., Collinsella spp., Dorea spp., Alistipes spp., Lactonifactor spp., Methanobrevibacter spp., Oscillospira spp., Sutterella spp., Eggerthella spp., Clostridioides spp., Turicibacter spp., Flavonifractor spp., Dialister spp., Bilophila spp., and Bulleidia spp, and/or combinations thereof.

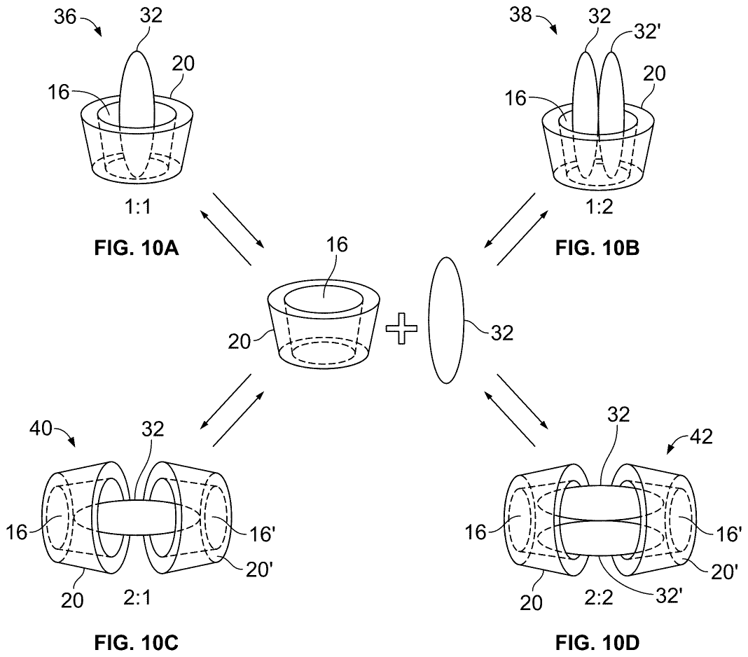

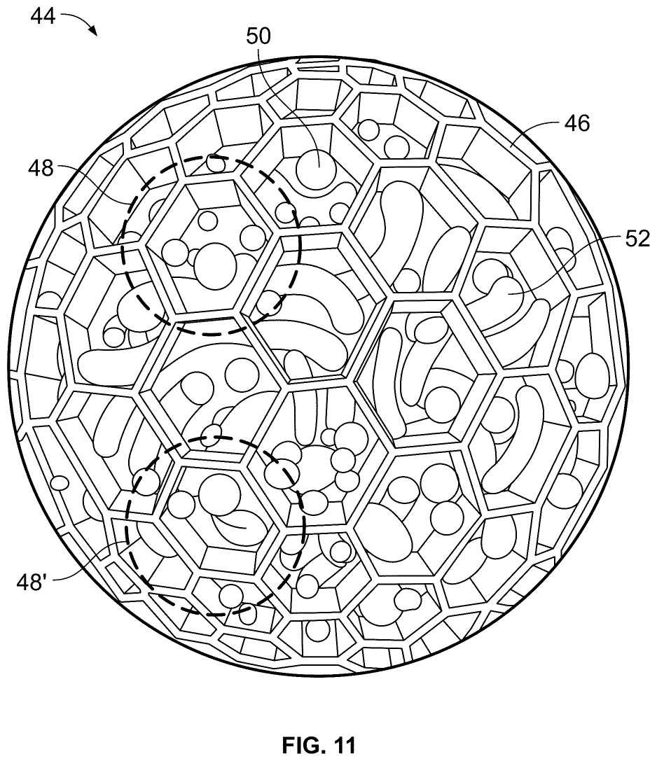

FIG. 10A shows a three-dimensional schematic representation of an example of a 1:1 configuration 36 for encapsulating (either entirely or partially) a single guest molecule 32 in a single, generic, hollow, cone-shaped, cyclodextrin molecule 20. In this example, guest molecule 32 is illustrated as being partially encapsulated inside of cavity 16 in cyclodextrin molecule 20. The guest molecule 32 may be a probiotic microorganism.

FIG. 10B shows a three-dimensional schematic representation of an example of a 1:2 configuration 38 for encapsulating (either entirely or partially) a pair of guest molecules 32 and 32′ in a single, generic, hollow, cone-shaped, cyclodextrin molecule 20. In this example, guest molecules 32 and 32′ are both illustrated as being partially encapsulated inside of cavity 16 in cyclodextrin molecule 20. The guest molecules 32 and 32′may be one or more probiotic microorganisms.

FIG. 10C shows a three-dimensional schematic representation of an example of a 2:1 configuration 40 for encapsulating (either entirely or partially) a single guest molecule 32 in an opposing pair of generic, hollow, cone-shaped, cyclodextrin molecules 20 and 20′. In this example, guest molecule 32 is illustrated as being partially encapsulated inside of both cavity 16 and 16′ in both cyclodextrin molecules 20 and 20′, respectively. The guest molecule 32 may be a probiotic microorganism.

FIG. 10D shows a three-dimensional schematic representation of an example of a 2:2 configuration 42 for encapsulating (either entirely or partially) a pair of guest molecules 32 and 32′ in an opposing pair of generic, hollow, cone-shaped, cyclodextrin molecules 20 and 20′. In this example, guest molecules 32 and 32′ are illustrated as being partially encapsulated inside of both cavities 16 and 16′ in both cyclodextrin molecules 20 and 20′, respectively. The guest molecules 32 and 32′ may be one or more probiotic microorganisms.

FIG. 11 shows a three-dimensional schematic representation of an example of an encapsulated probiotic assembly 44 comprising multiple probiotic microorganisms 50 and 52 encapsulated in a spherical, biocompatible structural cage 46. Structural cage 46 may be made of a biocompatible encapsulation compound, for example: a polysaccharide, liposome, alginate, chitosan, gelatin, polyvinyl alcohol, or cyclodextrin compound, and/or combinations thereof. Encapsulated probiotic assembly 44 may encapsulate multiple types of guest molecules, such as a spherical molecule 50 and/or an elongated, tubular molecule 52. Encapsulated probiotic assembly 44 may comprise a cyclodextrin cage 46 with multiple, interconnected, cyclodextrin inclusion complexes 48, 48′, etc. The multiple interconnected, cyclodextrin inclusion complexes 48, 48′, etc. may form a porous, honeycomb-like structural cage with the interstitial spaces filled with one or more guest molecules, as illustrated in this embodiment.

Referring still to FIG. 11, structural cage 46 may comprise multiple interconnected or linked (e.g., polymerized) cyclodextrin molecules (not shown individually), which create a protective environment around the encapsulated probiotic microorganisms 50 and 52. This encapsulation method and resulting structure 44 protects probiotic microorganisms 50 and 52 from external environmental factors, such as pH variation, enzymatic degradation, and moisture, thereby enhancing their stability and viability to facilitate the controlled release of probiotic microorganisms 50 and 52, which ensures their survivability and delivery to desirable target locations within the gastrointestinal tract of a human or animal host. This embodiment illustrates the functional arrangement between structural cage 46 (e.g., cyclodextrin-based cage 46) and the encapsulated probiotic microorganisms 50 and 52, without limitation to the specific configuration, structural pattern, or overall shape of structural cage 46. In this example, structural cage 46 has a “buckyball” shape, but other shapes (e.g., ellipses or cylinders) may alternatively be used.

In an embodiment, a cyclodextrin inclusion complex may have a guest molecule-to-cyclodextrin ratio, R, that ranges from about 0.1:1 to about 5:1. In an embodiment, a cyclodextrin inclusion complex may have a guest molecule-to-cyclodextrin ratio, R, that ranges from about 0.2:1 to about 2:1. In another embodiment, the guest molecule-to-cyclodextrin ratio, R, may range from about 0.5:1 to about 1:1.

In an embodiment, a solvent with a significant positive log(P) value, such as benzyl alcohol or limonene, may be used to enhance the complexation and stabilization of a wide range of guest molecules, including probiotic bacteria microorganisms, in large-particle cyclodextrin inclusion complexes.

In an embodiment, a cyclodextrin structure may be derivatized, with, e.g., hydroxypropyl groups. In embodiments in which a more hydrophilic guest (i.e., having a smaller log(P) value) is used, α-cyclodextrin may be used (i.e., alone or in combination with other types of cyclodextrins) to improve the encapsulation of the guest molecule in a cyclodextrin cage. For example, a combination of one or more α-cyclodextrin and one or more β-cyclodextrin molecules may be used in embodiments employing relatively hydrophilic guest molecules to improve the formation of large-particle cyclodextrin inclusion complexes.

In an embodiment, a guest molecule may comprise a probiotic microorganism.

The detailed description and the drawings or figures contained herein are supportive and descriptive of the present teachings, but the scope of the present teachings is defined solely by the claims. While some of the best modes and other embodiments for carrying out the present teachings have been described in detail, various alternative designs and embodiments exist for practicing the present teachings defined in the appended claims. All embodiments and examples disclosed herein are non-limiting embodiments and non-limiting examples. The words “a”, “an”, “the”, “at least one”, and “one or more” are used interchangeably to indicate that at least one of the items is present. The modifier “about” means that a specified variable has a range (tolerance) of no more than +/−10 % of the stated value of the specified variable. Moreover, words of approximation, such as “about,” “almost,” “substantially,” “generally,” “approximately,” and the like, may each be used herein to denote “at, near, or nearly at,” or “within 0-10% of,” or “within acceptable manufacturing tolerances,” or any logical combination thereof, for example.

What is claimed is:

1. A method of encapsulating one or more probiotic microorganisms, comprising:
(a) combining the one or more probiotic microorganisms with a solution, a suspension, or a medium comprising a biocompatible encapsulation compound; and
(b) encapsulating the one or more probiotic microorganisms at least partially within the biocompatible encapsulation compound to form an encapsulated probiotic assembly.

2. The method of claim 1, wherein step (b) comprises encapsulating the one or more probiotic microorganisms completely within the biocompatible encapsulation compound.

3. The method of claim 1, further comprising:
(c) processing the encapsulated probiotic assembly into a stable form suitable for at least one of oral, nasal, or rectal administration to a human host or an animal host.

4. The method of claim 1, wherein the biocompatible encapsulation compound is selected from the group consisting of cyclodextrins, polysaccharides, liposomes, alginates, chitosan, gelatin, synthetic polymers, hybrid compounds, polyvinyl alcohols, and combinations or derivatives thereof.

5. The method of claim 1, wherein the one or more probiotic microorganisms are selected from the group consisting of lactic acid bacteria, spore-forming bacteria, yeast-based probiotics, genetically engineered probiotic strains, methanogens, commensal gut bacteria, and combinations thereof.

6. The method of claim 1,
wherein the encapsulated probiotic assembly provides enhanced stability and bioavailability of the one or more probiotic microorganisms during storage and after ingestion by delaying release of the one or more probiotic microorganisms when passing through an upper gastrointestinal tract of a human host or an animal host, and
wherein the encapsulated probiotic assembly provides protection of the one or more probiotic microorganisms against environmental stressors selected from the group consisting of: heat, moisture, oxygen, pH variability, oxidative conditions, salinity, light, enzymatic activities, metal ions, free radicals, competing organisms, mechanical stresses, and combinations thereof.

7. The method of claim 1, further comprising
(c) administering an effective amount of the encapsulated probiotic assembly to a human or animal host via an oral, nasal, or rectal route.

8. The method of claim 1, further comprising using a solvent with a partition coefficient sufficient to enhance complexation and stabilization of the one or more probiotic microorganisms encapsulated at least partially within the biocompatible encapsulation compound.

9. The method of claim 8, wherein the solvent comprises one or more of ethyl alcohol, glycerol, dimethyl sulfoxide, or limonene, and combinations thereof.

10. The method of claim 1, wherein the one or more probiotic microorganisms are selected from the group consisting of: Lactobacillus spp., Bifidobacterium spp., Saccharomyces spp., Escherichia spp., Streptococcus spp., Bacillus spp., Enterococcus spp., Propionibacterium spp., Clostridium spp., Akkermansia spp., Faecalibacterium spp., Roseburia spp., Veillonella spp., Pediococcus spp., Leuconostoc spp., Lactococcus spp., Bacteroides spp., Eubacterium spp., Ruminococcus spp., Blautia spp., Parabacteroides spp., Desulfovibrio spp., Allobaculum spp., Megasphaera spp., Prevotella spp., Oscillibacter spp., Phascolarctobacterium spp., Coprococcus spp., Anaerostipes spp., Butyrivibrio spp., Collinsella spp., Dorea spp., Alistipes spp., Lactonifactor spp., Methanobrevibacter spp., Oscillospira spp., Sutterella spp., Eggerthella spp., Clostridioides spp., Turicibacter spp., Flavonifractor spp., Dialister spp., Bilophila spp., Bulleidia spp., and other bacterial, yeast, fungal, or methanogenic organisms that exhibit probiotic properties, including genetically engineered or modified strains designed to enhance therapeutic or functional effects, and/or combinations thereof.

11. The method of claim 1, further comprising, after step (b):
(c) subjecting the encapsulated probiotic assembly to one or more stabilization or drying processes selected from the group consisting of freeze-drying, spray-drying, vacuum-drying, cross-linking, and coating with a protective polymer, and combinations thereof; and
thereby obtaining a dried powder form of the encapsulated probiotic assembly.

12. The method of claim 11, further comprising after step (c):
(d) formulating the dried powder form of the encapsulated probiotic assembly into a delivery form comprising a capsule, powder, granule, suspension, tablet, gel, gummy, spray, or liquid delivery form.

13. The method of claim 12, further comprising after step (d):
(e) administering an effective amount of the delivery form of the encapsulated probiotic assembly to a human host or an animal host; and
wherein the encapsulated probiotic assembly provides at least one of controlled release, enhanced stability, and/or targeted delivery of the one or more probiotic microorganisms.

14. The method of claim 13, step (e) includes the encapsulated probiotic assembly surviving passage through an upper gastrointestinal tract of a human or an animal host without prematurely releasing the one or more probiotic microorganisms from the encapsulated probiotic assembly, thereby delivering the one or more probiotic microorganisms to at least one of a lower gastrointestinal tract, jejunum, ileum, colon, cecum, and a large intestine of a human host or an animal host.

15. The method of claim 1,
wherein the biocompatible encapsulation compound comprises one or more cyclodextrin molecules; and
wherein step (a) further comprises combining the one or more cyclodextrin molecules with the one or more probiotic microorganisms by using a process selected from the group consisting of co-precipitation, lyophilization, freeze-drying, spray-drying, vacuum-drying, kneading, solvent evaporation, nanoparticle formation, and combinations thereof to form a cyclodextrin inclusion complex.

16. The method of claim 15, wherein the one or more cyclodextrin molecules are selected from the group consisting of α-cyclodextrin, β-cyclodextrin, γ-cyclodextrin, derivatives of cyclodextrin, derivatized forms of cyclodextrin, and combinations thereof.

17. The method of claim 15, further comprising:
promoting growth and metabolic activity of the one or more probiotic microorganisms by releasing the one or more cyclodextrin molecules as a prebiotic agent from the encapsulated probiotic assembly inside of a human host or an animal host; and
wherein the prebiotic agent enhances clinical efficacy for treating or managing one or more of: dry eye disease, cardiovascular health, diabetes, dementia, obesity, skin health, oral health, or wound care, and combinations thereof.

18. A composition, comprising:
a pharmaceutically acceptable carrier; and
an encapsulated probiotic assembly combined with the pharmaceutically acceptable carrier;
wherein the composition further comprises:
a biocompatible encapsulation compound selected from the group consisting of cyclodextrins, derivatized cyclodextrins, liposomes, polysaccharides, alginate, chitosan, gelatin, polyvinyl alcohol, and combinations thereof; and
one or more probiotic microorganisms encapsulated at least partially within the biocompatible encapsulation compound; and
wherein the encapsulated probiotic assembly provides at least one of controlled release, enhanced stability, and targeted delivery of the one or more probiotic microorganisms.

19. The composition of claim 18,
wherein the biocompatible encapsulation compound comprises a cyclodextrin molecule or a derivatized cyclodextrin compound with one or more functional groups selected from the group consisting of methyl, ethyl, acetyl, hydroxypropyl, carboxymethyl, and sulfonate, and combinations thereof, which are selected to enhance at least one of solubility, stability, and bioavailability; and
wherein the one or more probiotic microorganisms are encapsulated at least partially within the cyclodextrin molecule or the derivatized cyclodextrin compound.

20. An encapsulated probiotic assembly, comprising:
a cyclodextrin molecule; and
a probiotic microorganism encapsulated at least partially within the cyclodextrin molecule; and
wherein the encapsulated probiotic assembly provides protection from environmental stressors and ensures targeted release in a lower gastrointestinal tract.

21. The encapsulated probiotic assembly of claim 20, wherein the cyclodextrin molecule is selected from the group consisting of α-cyclodextrin, β-cyclodextrin, γ-cyclodextrin, a derivative of cyclodextrin, and a derivatized form of cyclodextrin, and combinations thereof.

22. The encapsulated probiotic assembly of claim 20,
wherein the encapsulated probiotic assembly is formulated into a delivery form suitable for oral, nasal, or rectal administration to a human or animal host; and
wherein the delivery form is selected from the group consisting of a capsule, powder, granule, suspension, tablet, gel, gummy, spray, and liquid delivery forms, and combinations thereof.

23. The encapsulated probiotic assembly of claim 22, wherein the delivery form comprises a pharmaceutically acceptable carrier or excipient selected from the group consisting of a stabilizer, binder, disintegrant, lubricant, coating agent, antioxidant, active ingredient, and combinations thereof.
